(19) United States
(12) Patent Application Publication
(10) Pub. No.: US 2009/0238328 A1
(43) Pub. Date: Sep. 24, 2009

(54) X-RAY TUBE WITH OSCILLATING ANODE

(75) Inventors: Peter Forthmann, Hamburg (DE); Claas Bontus, Hamburg (DE); Thomas Köhler, Norderstedt (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/299,477

(22) PCT Filed: Apr. 25, 2007
(86) PCT No.: PCT/IB2007/051525

§ 371 Date: Nov. 4, 2008

(30) Foreign Application Priority Data
May 5, 2006 (EP) 06113541.4

(51) Int. Cl. A61B 6/00; H01J 35/00; H01J 35/30

(52) U.S. Cl. 378/14; 378/126; 378/137

(57) ABSTRACT

It is described an X-ray tube (205), in particular for use in computed tomography, comprising an electron source (250), for generating an electron beam (255), an electron deflection device (256) for deflecting the generated electron beam (255), a control unit (257) being coupled to the electron deflection device (256) for spatially controlling the deflection, and an anode (206), which is arranged such that the electron beam (255) impinges onto a focal spot of a surface of the anode (206). Thereby the anode (206) is movable along a z-axis in an oscillating manner, the surface of the anode (206) is oriented oblique with respect to the z-axis, and the control unit (257) is adapted to spatially control the focal spot (255 a) in such a manner that the focal spot moves essentially in a discrete manner between a first focal spot position (106a, 406a) having a first z-coordinate and a second focal spot position (106b, 406b) having a second z-coordinate being different from the first z-coordinate.

The present invention relates to an X-ray generating tube, which is adapted to generate X-ray originating from at least two spatially different focal spots. In particular, the present invention relates to an X-ray tube being used for computed tomography.

The present invention further relates to a computed tomography system being equipped with such an X-ray generating tube.

Further, the present invention relates to a method for operating an X-ray generating tube.

In some circumstances, it is desirable to provide a computed tomography (CT) apparatus with an X-ray source, which is capable of rapidly shifting a focal spot emitting X-rays from one place to another with respect to the patient being examined. It has been proposed to effect such shifting by electromagnetic or electrostatic deflection of the electron beam of the X-ray tube.

U.S. Pat. No. 4,002,917 and U.S. Pat. No. 4,010,371 disclose various CT arrangements in which such electron beam deflection is used to shift radiation paths laterally across the examined slice of a patient's body, longitudinally of said slice, or to hold the radiation in a certain disposition relative to the patient despite a physical rotation of the X-ray tube around the patient.

U.S. Pat. No. 4,162,420 discloses an X-ray tube including an envelope enclosing a flat-edged anode disc, which is rotatable and axially relocatable. The X-ray tube further encloses an electron beam source for projecting electrons along a beam axis toward the edge of the anode disc. The beam source is disposed to direct its beam at an acute angle of incidence to the edge of the anode disc and to produce X-rays, which are transmitted through a window in the envelope. The anode is elastically supported by means of two springs, wherein a first spring is attached at an upper end of an anode shaft and a second spring is attached at a lower end of the anode shaft. Thereby, the anode may be linearly shifted in an oscillating manner with respect to the envelope.

U.S. Pat. No. 4,107,563 discloses an X-ray generating tube, which is especially suitable for to be used in a CT apparatus. The X-ray generating tube comprises a rotating anode, which can be linearly shifted along a rotational axis of the anode in an oscillatory manner. The anode oscillation is realized by means of a so-called figure-of-eight groove, which is formed at a shaft of the rotating anode and which mechanically interacts with pegs being provided at a bearing of the rotating shaft. When the anode is shifted with respect to an envelope of the X-ray tube, a focal spot representing the origin of the generated X-ray is also moved with respect to the envelope. The described X-ray generating tube has the disadvantage that the oscillatory movement is directly connected with the rotational movement of the anode such that only a continuous displacement of the focal spot is possible. However, there are applications in particular in the field of CT, which require a fast switching of an X-ray focal spot between a first focal spot position and a second focal spot position.

There may be a need for an improved X-ray tube, which allows for a fast switching of an X-ray focal spot between a first focal spot position and a second focal spot position.

This need may be met by the subject matter according to the independent claims. Advantageous embodiments of the present invention are described by the dependent claims.

According to a first aspect of the invention there is provided an X-ray tube, in particular for generating X-rays being used for computed tomography. The provided X-ray tube comprises (a) an electron source, adapted for generating an electron beam projecting along a beam axis, (b) an electron deflection device for deflecting the generated electron beam, (c) a control unit being coupled to the electron deflection device for spatially controlling the beam axis and (d) an anode, which is arranged within the beam axis such that the electron beam impinges onto a focal spot of a surface of the anode. Thereby, the anode is movable along a z-axis in an oscillating manner, the surface of the anode is oriented oblique with respect to the z-axis, and the control unit is adapted to spatially control the focal spot in such a manner that the focal spot moves essentially in a discrete manner between a first focal spot position having a first z-coordinate and a second focal spot position having a second z-coordinate being different from the first z-coordinate.

This aspect of the invention is based on the idea that an essentially discrete switching of the focal spot between two different z-positions can be achieved even if there is a continuous and non-discrete oscillating movement of the anode. Thereby, the focal spot is moved over the surface of the anode in such a manner that the two focal spots have different radial distances with respect to the z-axis. Since the surface of the anode is oriented oblique with respect to the z-axis the radial focal spot movement caused by the electron deflection device also contributes to the variation of the focal spot along the z-direction. Thereby, by adequately operating the anode movement and the control unit in a synchronized manner, the contribution of the moving anode to the focal spot movement along the z-direction and the contribution of the electron beam deflection to the focal spot movement along the z-direction can be superimposed in such a manner that an essentially discrete switching of the focal spot along the z-direction may be achieved. This even holds if the anode movement and/or the electron beam deflection are not discrete. In other words, the electron beam deflection may compensate for a non-discrete movement of the anode.

By contrast to a focal spot displacement by means of the electron deflection device only, the described combined focal spot displacement being based on both the movement of the anode and the radial deflection of the electron beam provides the advantage that the difference of the radial distance of the two focal spots with respect to the z-axis is much smaller. Therefore, when operating the X-ray tube in a discrete focal spot switching mode the radial distance between the corresponding focal spot and an object being placed outside of the z-axis varies only slightly. This has the advantage that in many applications the radial focal spot movement may be neglected in a good approximation.

In particular when the described X-ray tube is used for increasing the sampling rate of digital X-ray attenuation data acquired e.g. by means of a computed tomography apparatus, an increased spatial resolution may be achieved within a wide region of interest. In this respect, an increase of the sampling rate may be achieved if for each projection angle of the X-ray source with respect to the object under examination two datasets are acquired. Thereby, a first dataset is acquired when the X-rays originate from the first focal spot and a second dataset is acquired when the X-rays originate from the second focal spot.

A further advantage of the focal spot displacement by means of both the electron deflection device and the mechanical motion of the anode is the fact that the requirements regarding the electron beam deflection unit are relaxed. This is based on the matter of fact that a major part of the z-movement of the focal spot is facilitated by the mechanical anode motion as compared to a focal spot z-movement caused solely by deflecting the electron beam.

According to an embodiment of the invention the anode is rotatable around the z-axis. This may provide the advantage that the concentration of the heat load of the anode may be reduced significantly because even when the electron beam generates only two discrete focal spots the heat load generated by a high-energy electron beam is distributed over a wide region on the anode surface.

According to a further embodiment of the invention the X-ray tube further comprises a spring element, which is arranged in between the anode and an envelope of the X-ray tube. This may provide the advantage that in particular a harmonic oscillation of the anode can be provided easily by means of simple elastic elements.

Preferably, the X-ray tube comprises at least two spring elements whereby a first spring element is attached to an upper portion of the anode and a second spring element is attached to an lower portion of the anode. This may provide the advantage that apart from providing an oscillatory movement the two springs may also contribute to a stable guidance of the anode parallel to the z-axis. Therefore, an unaccepted tilting of the anode may be prevented in a simple and effective manner.

It has to be pointed out that the spring element may be realized by mechanical and/or by electric respectively magnetic devices. For instance magnetic spring elements have the advantage that an abrasion or deterioration is negligible.

According to a further embodiment of the invention the X-ray tube further comprising a drive means, which is coupled to the anode in order to generate and/or to maintain an oscillatory movement of the anode. The drive means may be coupled mechanically and/or magnetically to the anode. A pure magnetic coupling has the advantage that the drive means may be realized without any movable mechanical parts.

According to a further embodiment of the invention the drive means is adapted to oscillate the anode with a frequency being essentially equal to a resonance frequency of the oscillating anode. This has the advantage that only little forces are needed to keep the anode oscillating at the desired frequency. Therefore, an essentially discrete switching of the focal spot positions may be realized without using complex mechanical apparatuses.

In this respect it is clear than apart from the mass respectively the weight of the anode also the spring constant of the spring element has a strong influence on the resonance frequency. Therefore, by taking into account the moving mass, the spring element or the spring elements have to be designed such that the resonant frequency of the system matches a predetermined focal spot frequency. In this context the focal spot frequency designates the frequency with which the focal spot is discretely switched between the first focal spot position and the second focal spot position and vice versa.

According to a further embodiment of the invention the drive means is adapted to oscillate the anode with a frequency being slightly bigger than a resonance frequency of the oscillating anode. This has the advantage that undesired anode vibrations may be reduced and that the anode may be oscillated in a piston like manner predominately along the z-axis.

Preferably, the oscillation frequency being slightly bigger than the resonance frequency is defined with respect to a curve exhibiting the resonance behavior of the oscillating system as a function of the driving frequency. Typically, the resonance behavior can be well approximated by a Lorenz curve having a maximum ω_{M} and a width Δω. Thereby, the width Δω strongly depends on the damping of the oscillating system.

Oscillating the anode with a frequency being slightly bigger than the resonance frequency means that the anode is oscillated with a frequency within a predetermined frequency range being defined by a lower frequency ω₁ and an upper frequency ω₂. Thereby, ω₁, may be equal to ω_{M} and ω₂ may be equal to ω_{M}+Δω. Preferably, ω₁ is equal to ω_{M}+Δω/20 and ω₂ is equal to ω_{M}+Δω/2. More preferably, ω₁ is equal to ω_{M}+Δω/10 and ω₂ is equal to ω_{M}+Δω/4.

According to a further aspect of the invention there is provided a computed tomography system comprising (a) a rotatable holder being rotatable around a rotation axis and (b) an X-ray tube according to any one of the embodiments described above, wherein the X-ray source is mounted at the rotatable holder in such a manner that the z-axis is oriented essentially parallel to the rotation axis. The computed tomography system further comprises (c) an X-ray detection device comprising a plurality of detector elements, the X-ray detection device being mounted at the rotatable holder opposite to the X-ray source with respect to the rotation axis.

This aspect of the invention is based on the idea that the above-described X-ray tube may be used advantageously for computed tomography wherein digital image reconstruction is based on the acquisition of at least two attenuation datasets wherein each dataset has been obtained with a different projection angle with respect to the object under examination. The spatial resolution of a reconstructed image strongly depends on the spatial resolution of the X-ray detection device, i.e. the spatial separation of the detector elements. When an essentially discrete switching of the focal spot position is carried out, for each projection angle, i.e. for each angular position of the rotational holder, two X-ray attenuation datasets may be acquired. Thereby, each voxel of the object under examination is penetrated with two different angles such that switching the focal spot position yields more detailed information regarding the attenuation respectively the absorption of the object under examination as compared to a data acquisition with one focal spot only.

According to an embodiment of the invention the first focal spot position is spatially separated from the second focal spot position in such a manner that a first fan of X-rays originating from the first focal spot, crossing the rotation axis and impinging on a row of various detector elements is interleaved with a second fan of X-rays originating from the second focal spot, crossing the rotation axis and impinging on the row of various detector elements.

Preferably, the computed tomography system allows for a predominantly symmetric interleaving such that the sampling rate of X-ray attenuation data may be doubled. Thereby, neighboring X-ray rays crossing the center of rotation are separated from each other by a distance being half of the distance between neighboring X-ray in the case when only one focal spot is used.

It has to be pointed out that in particular when the two focal spots have predominantly the same or at least a similar radial distance with respect to the z-axis, the so-called half-row sampling, which corresponds to a symmetric interleaving, might be realized not only within a region corresponding to a small section of the rotational axis. The symmetric interleaving might rather be realized within a wide region along the rotation axis.

It has to be mentioned that it is not necessary that the computed tomography system employs an X-ray tube which generates a fan beam. The computed tomography system might also take benefit from a cone beam geometry wherein a two dimensional detector array is used in order not only to detect X-rays crossing the rotation axis but also to detect X-rays passing the rotation axis. Thereby, the interleaving being symmetric for X-rays crossing the rotation axis might degenerate with an increasing distance between the rotation axis and the X-ray passing the rotation axis. However, as compared to a single focal spot X-ray tube the sampling rate of X-ray attenuation data with the described dual focal spot X-ray tube will anyway be increased significantly such that images with a higher spatial resolution may be reconstructed. A further advantage compared to a single focal spot X-ray tube is the fact that so-called splay or windmill artifacts may be reduced.

According to a further aspect of the invention there is provided a method for operating an X-ray tube, in particular for operating an X-ray tube being used for computed tomography. The provided method comprises (a) moving an anode along a z-axis in an oscillating manner, wherein the anode comprises a surface being oriented oblique with respect to the z-axis, (b) directing an electron beam being emitted from an electron source along a beam axis such that the electron beam impinges onto a focal spot of the surface and (c) spatially controlling the beam axis by means of an electron deflection device in such a manner that the focal spot moves essentially in a discrete manner between a first focal spot position having a first z-coordinate and a second focal spot position having a second z-coordinate being different from the first z-coordinate.

This aspect of the invention is based on the idea that by combining two movements namely the oscillating movement of the anode along the z-axis and a radial variation of the focal spot on the surface being oriented oblique with respect to the z-axis an essential discrete z-switching of the focal spot may by achieved even if the at least one of the movements is carried out in a non discrete manner. Thereby, a discrete focal spot switching may be realized without any mechanical step-wise motion. This has the advantage that the essential discrete X-ray focal point switching might be realized with a very simple mechanical system, which need not to be designed such stable that the system is capable of withstanding abrupt momentum transfers or jerky leaps caused by a stepwise motion of the anode.

According to an embodiment of the invention the first focal spot position is spatially separated from the second focal spot position in such a manner that a first fan of X-rays originating from the first focal spot, crossing the rotation axis and impinging on a row of various detector elements is interleaved with a second fan of X-rays originating from the second focal spot, crossing the rotation axis and impinging on the row of various detector elements.

Preferably, the focal spot variation allows for a symmetric interleaving such that the sampling rate of X-ray attenuation data may be doubled. As has already been mentioned above, in the case of symmetric interleaving neighboring X-ray rays crossing the center of rotation are separated from each other by a distance being half of the distance between neighboring X-ray originating from a single focal spot only.

It has to be pointed out that in particular when the two focal spots have predominantly the same or at least a similar radial distance with respect to the z-axis, the symmetric interleaving might be realized not only within a small section of the rotation axis. The symmetric interleaving might rather be realized within a wide region along the rotation axis.

According to a further embodiment of the invention the anode is moved in a sinusoidal manner. This may provide the advantage that the anode carries out a smooth harmonic motion, which causes only a comparatively small momentum transfer to a suspension for the anode. This in turn may provide the advantage that the essential discrete X-ray focal point switching might be realized with a very simple mechanical system, which need not to be designed very stable.

It has to be noted that embodiments of the invention have been described with reference to different subject matters. In particular, some embodiments have been described with reference to apparatus type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered to be disclosed with this application.

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

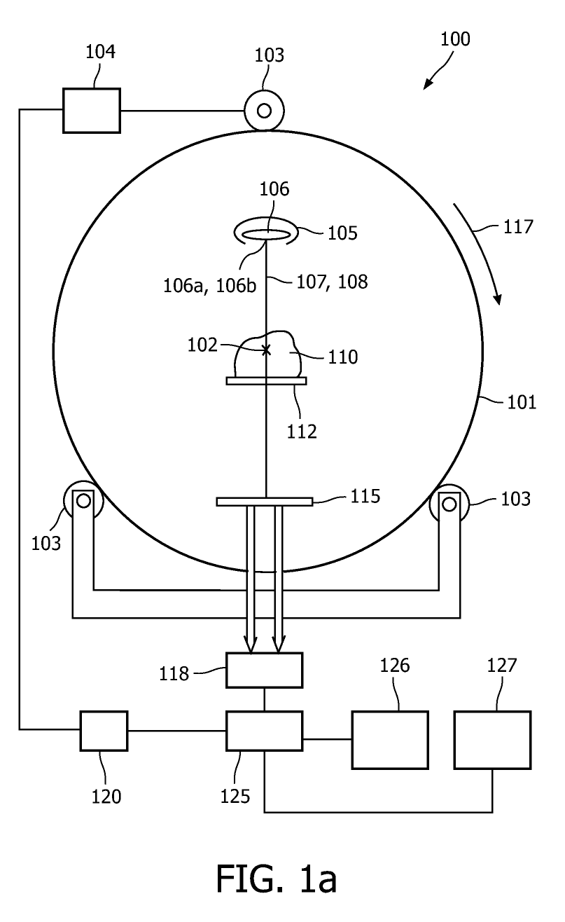

FIG. 1a shows a CT system according to a preferred embodiment of the invention in a simplified cross sectional view oriented perpendicular to a rotational axis.

FIG. 1b shows the X-ray beams originating from two different focal spots of the X-ray source of the CT system shown in FIG. 1a in a simplified cross sectional view oriented parallel to the rotational axis.

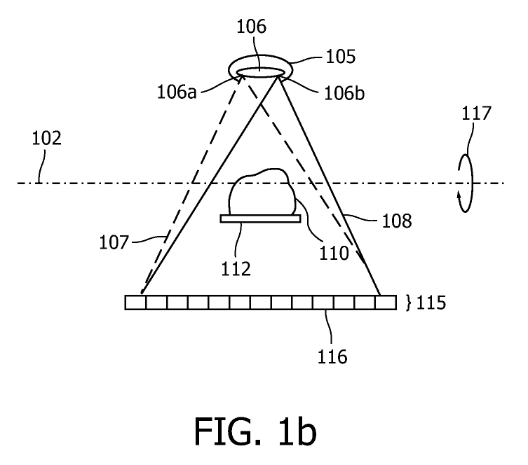

FIG. 2 shows an X-ray generating tube comprising an oscillating anode and an electron beam deflection unit.

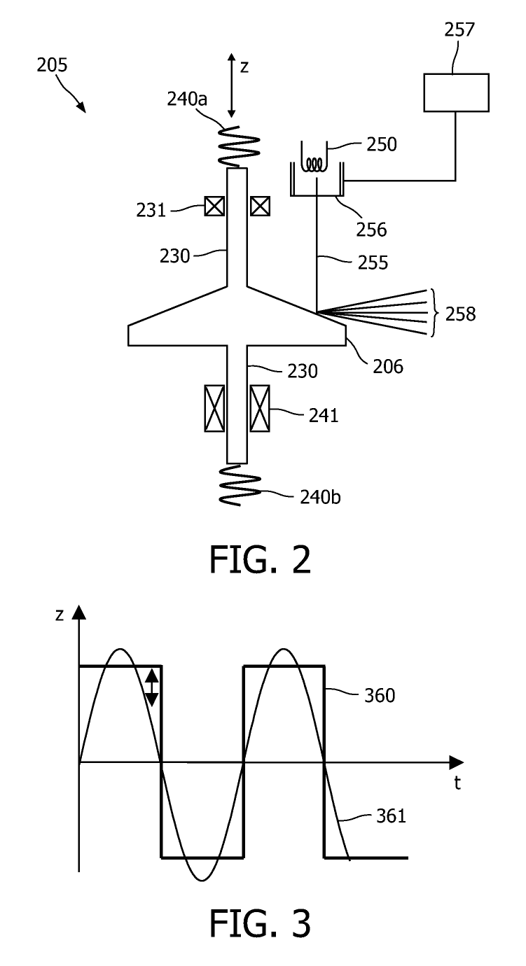

FIG. 3 shows a diagram depicting the discrepancy between an ideal step wise variation of the focal spot along the z-axis and an harmonic mechanic motion of the anode.

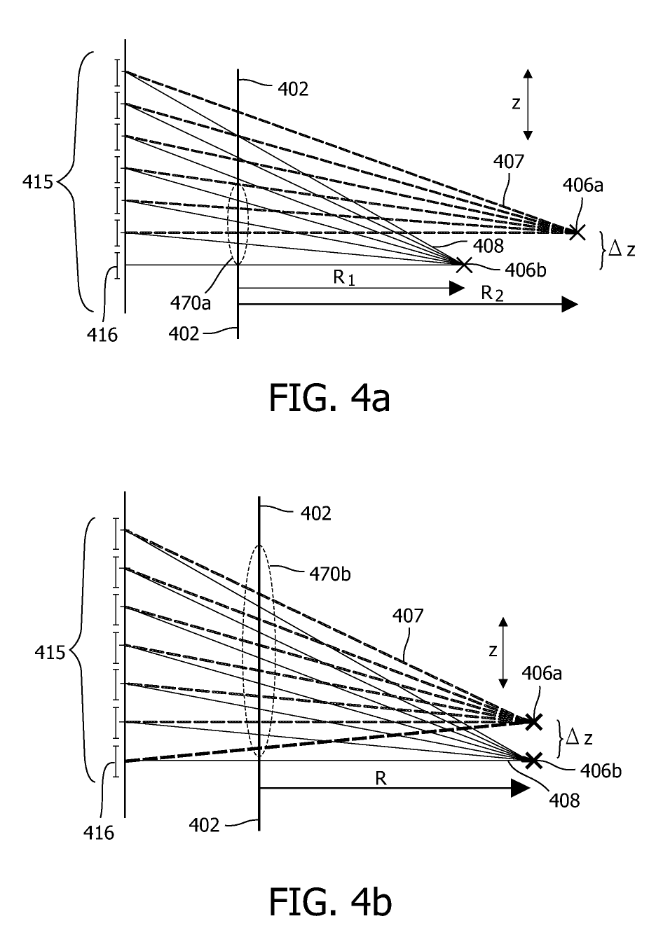

FIGS. 4a and 4b illustrate the influence of a radially varying focal spot position on the interleaving between a first fan of X-rays originating from a first focal spot and a second fan of X-rays originating from a second focal spot.

The illustration in the drawing is schematically. It is noted that in different figures, similar or identical elements are provided with the same reference signs or with reference signs, which are different from the corresponding reference signs only within the first digit.

FIG. 1a shows a CT scanner 100 comprising a rotatable holder 101 in which an X-ray source 105 and an X-ray detection device 115 are incorporated. The holder 101 is rotated around a rotational axis 102 by means of a drive motor 104 and a drive mechanism. The drive mechanism is symbolized by means of three drive rollers 103. The rotation of the holder 101 may be accomplished in a continuous or in a stepwise manner.

The CT scanner 100 further comprises a table 112, which is arranged such that an object under examination 110 may be positioned in the center of the holder 101. The table 112 may be movable with respect to the gantry 101 in a direction parallel to the rotational axis 102 such that different portions of the object under examination 110 can be examined.

The X-ray detection device 115 contains at least one row of interconnected detector elements, wherein the row extends parallel to the rotational axis. The detector elements can all be read out separately via a preamplifier 118 and a data processing device 125. The data processing device 125 is capable of converting the measured detector signals. By measuring attenuation signals under a variety of different projection or viewing angles of the X-ray source 105 with respect to the object 110, the data processing device 125 is capable of reconstructing a three dimensional representation of the object 110. The reconstructed images may be outputted by means of a monitor 126 and/or by means of a printer 127.

The data processing device 125 is further coupled with a motor control unit 120, which is used for controlling the movement of the rotatable holder 101 in a rotation direction indicated by an arrow 117.

The X-ray source 105 is an X-ray tube comprising an anode 106. The anode 106 is elongated in a direction parallel to the rotational axis 102. An electron beam emitted by a cathode, which is not indicated here, can be directed discretely onto one of two X-ray focal spots, onto a first X-ray focal spot 106a and onto a second X-ray focal spot 106b. Preferably, these two focal spots 106a and 106b are oriented as close as possible in a row parallel to the rotational axis 102 such that in FIG. 1a the two focal spots 106a and 106a cannot be visually discriminated from each other. As a consequence, also a first radiation beam 107 originating from the first X-ray focal spot 106a and a second radiation beam 108 originating from the first X-ray focal spot 106b can also not be discriminated from each other.

The data processing device 125 is further coupled with an electronic control unit (not depicted) in order to provide for a synchronization between the data acquisition and spatially switching the electron beam between the two focal points 106a and 106b.

FIG. 1b shows an enlarged representation of the X-ray tube 105, the object under examination 110 and the X-ray detection device 115 in a cross sectional view parallel to the rotational axis 102. The two focal spots 106a and 106b are oriented in a row essentially parallel to the rotational axis 102. A discrete switching of the X-ray focal spot between the two focal spots 106a and 106b has the effect that the object 110 is sequentially illuminated with the X-ray beams 107 and 108 under slightly different projection angles. Therefore, each detector element 116 of the X-ray detection device 115 can detect two different X-ray attenuation line integrals, a first line integral extending between the first focal spot 106a and the detector element 116 and a second line integral extending between the second focal spot 106b and the detector element 116. As a consequence, for each projection angle of the system X-ray source 105 and X-ray detection device 115 with respect to the object 110 i.e. for each angular position of the gantry 101 two different datasets may be acquired which can be combined in an appropriate manner such that the spatial resolution of the CT scanner 100 can be enhanced.

FIG. 2 shows an X-ray tube 205, which is adapted to generate X-rays originating from different X-ray focal spots. The X-ray tube 205 comprises an anode 206 having a shaft 230. The shaft 230 is guided in such a manner that the shaft 230 may be both shifted linearly along a z-axis and rotated around the z-axis. A rotational drive 231 is provided in order to allow for a rotational movement of the anode 206. In order to allow for a linear movement of the anode 206 an oscillatory drive 241 is provided. Both drives 231 and 241 may interact with the shaft 230 by means of a mechanical and/or a magnetic interaction.

The X-ray tube 205 further comprises an electron source 250, which is arranged laterally with respect to the z-axis. According to the embodiment described here, the electron source is a hot cathode 250, which during operating generates an electron beam 255. The electron beam impinges onto a top surface of the anode 206. Thereby, a focal spot is defined. The top surface is oriented oblique with respect to the z-axis such that from the focal spot an X-ray beam 258 projects radially outwards from the z-axis.

In order to control the exact position of the focal spot the X-ray tube 205 further comprises an electron deflection device 256, which is adapted to deflect the electron beam 255. The electron deflection device 256 may be realized by known electron optic elements such as e.g. magnetic lenses. The electron deflection device 256 is coupled to a control unit, which provides the necessary electric signals to the electron deflection device 256.

Further, the X-ray tube 205 comprises two spring elements 240a and 240b, which are attached to an upper end of the shaft 230 and to a lower end of the shaft 230, respectively. The spring elements, which may be realized by mechanical and/or magnetic means, are also attached to a not shown support structure of the X-ray tube 205. The support structure may be for instance an envelope of the X-ray tube 205.

The system anode 206 and the two spring elements 240a and 240b represent an harmonic oscillator having a resonance frequency which is given by the mass of the anode and by the spring constants of the spring elements 240a and 240b. Therefore, the anode 206 will preferably exhibit a sinusoidal motion along the z-axis. However, it is clear that also a non-perfect sinusoidal movement of the anode 206 may be enforced by the oscillatory drive 241. However, the stronger the discrepancy between the real movement and a perfect sinusoidal movement is, the bigger are the mechanical forces which act on the support structure of the X-ray tube. This has the effect that it will be become very difficult to control a movement deviating strongly from a harmonic motion.

However, when the described X-ray tube 205 is supposed to be used as a dual focus X-ray tube it is desirable that the z-coordinate of the focal spot does not move in a continuous manner. In order to acquire two different X-ray attenuation datasets under slightly different projection angles and to reduce smearing effects in between these two datasets it is rather desirable that the focal spot moves at least essentially in a discrete manner between two focal spots.

FIG. 3 shows a diagram depicting the discrepancy between an ideal step wise variation 360 of the focal spot along the z-axis and an harmonic z-motion 361 of the anode 206. This discrepancy is illustrated by a double-headed arrow. It can be recognized that the discrepancy periodically varies in a synchronized manner with the harmonic motion. Of course, the overall discrepancy will be minimized when the period of the harmonic motion is selected such that it is equal to the period of the step wise z-motion 360.

According to the embodiment described here the discrepancy between the step wise motion 360 and the harmonic motion 361 is compensated by an appropriate deflection of the electron beam 255 such that also a radial movement of the focal spot contributes to a variation of the z-coordinate of the focal spot. In other words, by adequately operating the anode movement and the radial movement of the focal spot in a synchronized manner, the contribution of the moving anode to the focal spot movement along the z-direction and the contribution of the radial electron beam deflection to the focal spot movement along the z-direction can be superimposed in such a manner that an essentially discrete switching of the focal spot along the z-direction may be achieved.

It has to be mentioned that in order to achieve a focal spot variation, which is discrete as much as possible, it might be preferable to generate an oscillatory movement of the anode 206, which slightly deviates from a perfect sinusoidal movement. Thereby, in order to generate an essentially discrete movement of the focal spot the contribution of the anode movement may be increased and the contribution of the radial electron deflection may be decreased.

By contrast to known techniques for a quasi discrete switching of an X-ray focal spot by means of electron beam deflection only, the described X-ray tube 205 has the advantage that the radial focal spot variation is reduced. In the following this advantage will be described with reference to the FIGS. 4a and 4b.

FIGS. 4a and 4b illustrate the influence of a radially varying focal spot position on the interleaving between a first fan of X-rays 407 originating from a first focal spot 406a and a second fan of X-rays 408 originating from a second focal spot 406b.

As can be seen from FIG. 4a, a variation of the focal spot position which occurs not only along the z-axis but which occurs also radially with respect to the z-axis has an unwanted side effect. Thereby, when varying the focal spot position with Δz the radial distance between the rotational axis 402 and the focal spot position changes from R₁ to R₂ or vice versa. This unwanted effect causes that an interleaving of X-rays 407 originating from the first focal spot 406a with X-rays 408 originating from the second focal spot 406b occurs within a small region 470a only. This region 470a extends along a comparatively short section of the rotational axis 402.

Interleaving, which is a known procedure in order to enhance the spatial resolution, is based on the fact that neighboring X-ray rays, which originate from different focal spots, which cross the rotational axis 402 and which impinge on the same detector element 416 of the X-ray detection device 415, are separated from each other by a distance being half of the distance between neighboring X-rays, which originate from one focal spot only and which impinge on neighboring detector elements 416. In the case of a symmetric interleaving the sampling rate of X-ray attenuation data may be doubled.

As can be seen from FIG. 4b, a variation of the focal spot position occurring predominately only along the z-axis has the advantage that the corresponding interleaving region 470b is much bigger than the reduced interleaving region 470a. Due to the constant radial position R of both focal spots 406a and 406b with respect to the rotational axis 402 a symmetric interleaving may be realized within the comparatively big region 470b extending along the z-axis.

It has to be pointed out that when using the above-described X-ray tube 205 one can achieve an essential step wise z-variation of the focal spot, whereby the radial variation of the focal spot position can be minimized. Therefore, the above-described X-ray tube 205 allows for an improved interleaving and as a consequence for acquiring X-ray attenuation data with an improved spatial resolution.

It has to be mentioned that although the enhanced interleaving effect has been described with reference to a fan beam wherein all rays cross the rotational axis 402, it is also possible to take benefit from a cone beam geometry wherein a two dimensional detector array is used in order to not only detect X-rays crossing the rotation axis but also to detect X-rays passing the rotation axis in a predetermined distance. Thereby, the interleaving being symmetric for X-rays crossing the rotation axis might degenerate with an increasing distance between the rotation axis and the X-ray passing the rotation axis. However, as compared to a single focal spot X-ray tube the sampling rate of X-ray attenuation data with the described dual focal spot X-ray tube will anyway be increased significantly such that X-ray images with a higher spatial resolution may be provided.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

### LIST OF REFERENCE SIGNS

100 computer tomography apparatus/CT scanner
101 rotatable holder/gantry
102 rotational axis
103 drive rollers
104 drive motor
105 X-ray source
106 anode
106a first X-ray focal spot
106b second X-ray focal spot
107 first radiation beam
108 second radiation beam
110 object under examination
112 table
115 X-ray detection device
116 detector elements
117 rotation direction
118 preamplifier
120 motor control unit
125 data processing device (incl. reconstruction unit)
126 monitor
127 printer
205 X-ray source/X-ray tube
206 anode
230 shaft
231 rotational drive
240a/b spring elements
241 oscillatory drive
250 electron source/hot cathode
255 electron beam
255a focal spot
256 electron deflection device
257 control unit
258 X-ray beam
360 ideal stepwise z-motion
361 harmonic z-motion of anode
402 rotational axis
406a first X-ray focal spot
406b second X-ray focal spot
407 first radiation beam
408 second radiation beam
415 X-ray detection device/row of detector elements 416
416 detector element
470a interleaving region (small)
470b interleaving region (big)
Δz focal spot variation along the z-axis
R₁ radial distance between the rotational axis 402 and the first focal spot 406a
R₂ radial distance between the rotational axis 402 and the second focal spot 406b
R radial distance between the rotational axis 402 and both focal spots 406a, 406b

1. An X-ray tube, in particular for generating X-rays being used for computed tomography, the X-ray tube (205) comprising
an electron source (250), adapted for generating an electron beam (255) projecting along a beam axis,
an electron deflection device (256) for deflecting the generated electron beam (255),
a control unit (257) being coupled to the electron deflection device (256) for spatially controlling the beam axis, and
an anode (206), which is arranged within the beam axis such that the electron beam (255) impinges onto a focal spot of a surface of the anode (206),
wherein
the anode (206) is movable along a z-axis in an oscillating manner,
the surface of the anode (206) is oriented oblique with respect to the z-axis, and
the control unit (257) is adapted to spatially control the focal spot in such a manner that the focal spot moves essentially in a discrete manner between a first focal spot position (106a, 406a) having a first z-coordinate and a second focal spot position (106b, 406b) having a second z-coordinate being different from the first z-coordinate.

2. The X-ray tube according to claim 1, wherein
the anode (206) is rotatable around the z-axis.

3. The X-ray tube according to claim 1, further comprising
a spring element (240a, 240b) which is arranged in between the anode (206) and an envelope of the X-ray tube (205).

4. The X-ray tube according to claim 3, further comprising
a drive means (241), which is coupled to the anode (206) in order to generate and/or to maintain an oscillatory movement of the anode (206).

5. The X-ray tube according to claim 4, wherein
the drive means (241) is adapted to oscillate the anode with a frequency being essentially equal to a resonance frequency of the oscillating anode (206).

6. The X-ray tube according to claim 4, wherein
the drive means (241) is adapted to oscillate the anode (206) with a frequency being slightly bigger than a resonance frequency of the oscillating anode (206).

7. A computed tomography system comprising
a rotatable holder (101) being rotatable around a rotation axis (102, 402),
an X-ray tube (105, 205) as set forth in claim 1, the X-ray tube (105, 205) being mounted at the rotatable holder (101) in such a manner that the z-axis is oriented essentially parallel to the rotation axis (102, 402),
an X-ray detection device (115, 415) comprising a plurality of detector elements (116, 416), the X-ray detection device (115, 415) being mounted at the rotatable holder (101) opposite to the X-ray tube (105, 205) with respect to the rotation axis (102, 402).

8. The computed tomography system according to claim 7, wherein
the first focal spot position (106a, 406a) is spatially separated from the second focal spot position (106b, 406b) in such a manner that a first fan of X-rays (107, 407) originating from the first focal spot (106a, 406a), crossing the rotation axis (102, 402) and impinging on a row of various detector elements (116, 416) is interleaved with a second fan of X-rays (108, 408) originating from the second focal spot (106b, 406b), crossing the rotation axis (102, 402) and impinging on the row of various detector elements (116, 416).

9. A Method for operating an X-ray tube (205), in particular for operating an X-ray tube being used for computed tomography, the method comprising
moving an anode (206) along a z-axis in an oscillating manner, wherein the anode (206) comprises a surface being oriented oblique with respect to the z-axis,
directing an electron beam (255) being emitted from an electron source (250) along a beam axis such that the electron beam (255) impinges onto a focal spot of the surface and
spatially controlling the beam axis by means of an electron deflection device (256) in such a manner that
the focal spot moves essentially in a discrete manner between a first focal spot position (106a, 406a) having a first z-coordinate and a second focal spot position (106b, 406b) having a second z-coordinate being different from the first z-coordinate.

10. The method according to claim 9, wherein
the first focal spot position (106a, 406a) is spatially separated from the second focal spot position (106b, 406b) in such a manner that
a first fan of X-rays (107, 407) originating from the first focal spot (106a, 406a), crossing the rotation axis (102, 402) and impinging on a row of various detector elements (116, 416) is interleaved with a second fan of X-rays (108, 408) originating from the second focal spot (106b, 406b), crossing the rotation axis (102, 402) and impinging on the row of various detector elements (116, 416).

11. The method according to claim 9, wherein
the anode (206) is moved in a sinusoidal manner.
